(19) United States
(12) Patent Application Publication
(10) Pub. No.: US 2006/0041274 A1
(43) Pub. Date: Feb. 23, 2006

(54) Modified laparoscopic instruments and methods of use

(76) Inventor: Li-Ming Su, Ellicott City, MD (US)

Correspondence Address: EDWARDS & ANGELL, LLP, P.O. BOX 55874, BOSTON, MA, 02205, US

(21) Appl. No.: 11/209,956

(22) Filed: Aug. 22, 2005

Related U.S. Application Data
(60) Provisional application No. 60/603,014, filed on Aug. 20, 2004.

(51) Int. Cl. A61B 17/28

(52) U.S. Cl. 606/205

(57) ABSTRACT

Instruments and methods for performing delicate surgical procedures. The instruments include dissectors and clamps having a modified angled, fine sized tips that are adapted for use in performing delicate surgical procedures, particularly nerve-sparing radical prostatectomy.

This application claims the benefit of U.S. Provisional Application Ser. No. 60/603,014 filed Aug. 20, 2004, the teachings of which are incorporated herein by reference in its entirety.

### FIELD OF THE INVENTION

The present invention relates to surgical devices, systems, and techniques adapted for performing fine dissection of delicate tissues, more particularly to devices, systems, and techniques for performing laparoscopic radical prostatectomy, and more specifically to surgical devices such as clamps and dissectors, for use in performing nerve-sparing laparoscopic radical prostatectomy.

### BACKGROUND OF THE INVENTION

Prostate cancer is the most commonly diagnosed cancer in men in the United States and is tied for the second leading cause of cancer death for males. With the widespread use of prostate-specific antigen (PSA) tests and digital rectal examinations for the early detection of prostate cancer, most new cases are being diagnosed at early and potentially curable stages.

Radical prostatectomy is a surgical technique that involves major surgery to remove prostate cancer. During the surgery, the entire prostate gland and some surrounding tissue are removed. Also, the pelvic lymph nodes may be sampled for a biopsy. The goal is to remove the cancer entirely and prevent its spread to other parts of the body. Radical prostatectomy can be performed using either an open surgical technique(s) or a laparoscopic surgical technique.

Open surgery or the open surgical technique can be performed by either a retropubic or perineal approach. Using the retropubic approach, an incision is made just below the navel and extends to just above the pubic bone. In the perineal approach, a smaller, curved incision is made between the anus and the base of the scrotum and the prostate is then removed from underneath the pubic bone.

Laparoscopic prostatectomy is a less invasive procedure that eliminates the need for making a large surgical incision to remove the prostate. As a result, the patient may experience less pain and scarring, faster recovery and less risk of infection. During a laparoscopic prostatectomy a telescopic instrument called a laparoscope is inserted into the abdomen through a small incision at the belly button. A camera attached to the laparoscope allows surgeons to view inside the abdomen and perform the surgery without having to make a large incision. Usually, four more small incisions are made in the abdomen to accommodate surgical instruments during surgery.

Each type of surgery also may be done as a nerve-sparing or non-nerve-sparing type of procedure. Non-nerve-sparing procedures remove the entire prostate, the tissue surrounding it, and the nerves responsible for erections, called the cavernous nerves. Nerve-sparing procedures are used to avoid damaging the cavernous nerves that run alongside the prostate and control erection (aka, neurovascular bundle). The preservation of the cavernous nerves during radical prostatectomy is a delicate surgical technique that is crucial in reducing the incidence of postoperative impotence in men suffering from prostate cancer. Such nerve-sparing procedures require meticulous tissue handling so as to maximize potency preservation.

While laparoscopic procedures are generally preferable over open surgery or open surgical procedures, radical retropubic prostatectomy (RRP) remains the reference standard for the surgical treatment of patients with clinically localized prostate cancer. Laparoscopic procedures are more complex and complications of laparoscopic procedures correlate highly to the level of surgeon experience and the instruments available to carry out such procedures. In particular, during such laparoscopic procedures, the various instruments are inserted through cannulas or sleeves that are inserted through small incisions to provide entry ports through which the instruments are passed.

The surgical instruments used in laparoscopic procedures are generally similar to those used in open surgical procedures except that they include an extension between the end of the instrument entering the surgical field (i.e., the operable end of the instrument) and the portion held by the surgeon. This arrangement, however, provides limited motion at the operable end of the device because such motion takes place through a cannula or sleeve, which results in inverted motion. Because most instruments are rigid, motion is limited to four degrees of freedom about the incision point as well as in and out of the incision. Laparoscopic procedures also make it difficult for the surgeon to accurately perceive the force and interaction between the instruments and internal structures. Further, while the surgeon may use his or her hands to manipulate and retract tissues during open surgical procedures, grasping devices must be used during laparoscopic procedures to hold and move one or more of a patient's organs or other tissue so the physician can carry out the desired surgery.

Thus, while laparoscopic procedures are generally preferable, such procedures present additional challenges that make the preservation of the cavernous nerves particularly difficult. Further, the techniques for cavernous nerve preservation during open radical prostatectomy, particularly retropubic, have undergone several decades of careful refinement.

Consequently, current laparoscopic instruments and methods for performing nerve-sparing radical prostatectomy do not adequately provide precise dissection and preservation of the cavernous nerves and, thus, increase the risk that the nerve bundles will be injured or traumatized. In particular, current laparoscopic devices include large, bulky dissectors and clamps that are cumbersome to use and do not allow for fine movements and dissection of delicate structures such as blood vessels and nerves. This can result in the inability to safely dissect and optimally preserve the cavernous nerves as well as other delicate structures. Further, current methods routinely utilize bipolar and monopolar electrocautery and ultrasonic shears to achieve hemostasis and facilitate dissection. Such methods can result in thermal injury to the cavernous nerves and nearby tissues.

Thus, there is a need for improved laparoscopic instruments to help facilitate preservation of the delicate cavernous nerves during laparoscopic radical prostatectomy. Such instruments and devices preferably would be specifically designed to allow for the delicate handling and preservation of tissues and the cavernous nerves, thereby reducing the risk of injury or trauma to the tissues and nerves, which in turn should increase the likelihood of preserving the patient's sexual function. The related methods would utilize such instruments or devices and further would avoid the use of any electrocautery, heat or electrical energy around the cavernous nerves. Such instruments, devices, and methods also would be adaptable for use in connection with open surgeries or open surgical procedures.

### SUMMARY OF THE INVENTION

The present invention features an improved surgical device(s) that provides precise dissection of tissues and other anatomical structures. Such a device is suitable for use in laparoscopic procedures on both humans and in veterinary procedures. While particularly suitable for laparoscopic procedures, it should be recognized that such a device also is adaptable so as to be used in connection with open surgical procedures. Further, the devices and methods are suitable for use in robot assisted laparoscopic procedures.

In one preferred embodiment, such a surgical device is designed for use in performing radical prostatectomy. Such procedures require gentle and meticulous handling and dissection of tissues, particularly in nerve-sparing procedures wherein the cavernous nerve bundles are dissected away from the prostate surface.

A device of the present invention comprises a generally elongate housing member having a proximal end and a distal end and a handle member located at the proximal end. The handle member can be used by a surgeon to grasp the device. The distal end comprises the operable end of the device and can be in the form of a grasper, retractor, clamp, dissector, scissors, forceps, biopsy punch, biopsy spoon, and hook as well as any other conventional surgical instrument that can be used in the surgical procedure.

In a preferred embodiment, the operable end comprises a clamp. In another preferred embodiment, the operable end comprises a dissector. The clamp and the dissector both include a proximal end, distal end, and a bend or curve there between (e.g., 60 and 90 degree dissectors). In preferred embodiments, the distal end of the clamp and dissector tapers to a diameter of less than that found in conventional clamps and dissectors. The bend provides an angle between the proximal end and the distal end that facilitates the surgeon's ability to access the surgical site, particularly for separation of the cavernous nerves/neurovascular bundle from the prostate.

FIG. 6 shows a schematic illustration of the dorsal vein complex, pelvic fascia, striated urethral sphincter, smooth musculature of the urethra, and neurovascular bundles, which must be dissected away from the prostate surface. The instruments and devices of the present invention, having a bend together with the tapered distal end provides finer and more gentle and meticulous dissection to be achieved when dissecting the delicate cavernous nerve bundles away from the prostate surface. Thus, the chance of preserving the cavernous nerves and reduce postoperative impotence is increased or optimized.

In an exemplary embodiment, a surgical instrument suitable for use in performing nerve-sparing radical prostatectomy is provided comprising an elongate shaft having a proximal end and a distal end, a handle at the proximal end of the shaft and an operable end at the distal end of the shaft. The operable end includes at least two arms that are disposed opposite each other, the two arms having inner surfaces in longitudinal engagement with each other. The operable end defines a proximal portion, a distal portion, and a curved portion between the proximal portion and distal portion, and the distal portion tapers to a diameter of less than about 2 mm. More preferably, the distal portion tapers to a diameter of less than about 1.5 mm, preferably less than about 1.4 mm, preferably less than about 1.3 mm, preferably less than about 1.2 mm, preferably less than about 1.1 mm, preferably less than about 1 mm, more preferably from about 0.5 mm to about 0.9 mm.

In an exemplary embodiment, the device comprises a very fine-tipped right-angled (i.e., 90 degree) clamp (0.8 mm) and curved (i.e., 60 degree) dissector (0.8 mm) for use in laparoscopic prostatectomy that reduces the damage to cavernous nerves during surgery. In further embodiments, such a dissector and/or clamp are used to dissect the fine cavernous nerve bundles from the prostate during laparoscopic radical prostatectomy.

In a preferred method, a combined antegrade and retrograde laparoscopic approach to neurovascular bundle dissection is used. Such a method includes dissection of the neurovascular bundle using a laparoscopic instrumentation including a fine-tipped right-angle clamp and a fine-tipped curved dissector. These instruments allow for meticulous tissue handling. Further, the methods of the present invention are preferably carried out without the use of electrocautery.

In one embodiment, instruments and techniques are provided wherein a nerve-sparing laparoscopic radical prostatectomy (LRP) technique replicates that of an anatomic nerve-sparing radical retropubic prostatectomy (RRP). In particular, the techniques utilize the modified dissectors and clamps, involve identifying pre-existing anatomic planes, and avoiding thermal injury near the nerves.

In further embodiments, there is featured a surgical instrument or device suitable for use in performing nerve-sparing radical prostatectomy. Such a surgical includes an elongate shaft having a proximal end and a distal end, a handle at the proximal end of the shaft, and an operable end at the distal end. The operable end includes at least two arms that are disposed opposite each other, the two arms having inner surfaces in longitudinal engagement with each other. The operable end also has a proximal portion, a distal portion, and a curved portion between the proximal portion and distal portion.

The distal portion tapers to a diameter of less than about 2 mm. In further embodiments, the distal portion tapers to a diameter at its smallest location(s) that is no greater than about 1.5 mm, preferably no greater than about 1.4 mm, preferably no greater than about 1.3 mm, preferably no greater than about 1.2 mm, preferably no greater than about 1.1 mm, preferably no greater than about 1 mm, preferably no greater than about 0.9 mm, preferably no greater than about 0.8 mm. In yet further embodiments, the distal portion tapers to a diameter at its smallest location that is between about 0.5 mm and 1 mm, more preferably between about 0.6 mm and about 0.9 mm, and more preferably between about 0.7 and 0.8 mm.

The handle of such an instrument/device of the present invention can further include one or more movable finger and thumb rings in connection with the arms or jaws. Such moveable finger and thumb rings are operably coupled to the arms or jaws such that when manipulation of the one or more finger and thumb rings changes the distance between inner surfaces of the arms or jaws.

According to another aspect of the present invention there is featured methods for performing nerve-sparing radical prostatectomy. Such methods include providing a surgical instrument of the present invention such as that herein described. In embodiments, the provided surgical instrument includes elongate shaft having a proximal end and a distal end, an operable end at the distal end of the shaft comprising at least two arms that are disposed opposite each other, the two arms having inner surfaces in longitudinal engagement with each other, the operable end having a proximal portion, a distal portion, and a curved portion between the proximal portion and distal portion, wherein the distal portion tapers to a diameter of less than about 2 mm.

Such method further includes inserting the distal portion of the operable end between the prostate and the cavernous nerves and manipulating the operable end distal portion so as to separate the prostrate from the cavernous nerves. In more particular embodiments such methods include providing the two arms with their inner surfaces in longitudinal engagement and manipulating the arms of the operable end so as to provide a distance between their inner surfaces, thereby separating the prostate from the cavernous nerves. Such methods also further includes dissecting the cavernous nerves from the prostate.

In further embodiments, such methods of the present invention further includes forming a plurality of incisions in the mid abdomen, inserting a plurality of laparoscopic sleeves through the incisions and inserting the surgical instrument into the surgical site via a laparoscopic sleeve. Using accepted surgical techniques, the inserted surgical instruments of the present invention are manipulated as herein described so as to dissect the cavernous nerves.

In yet a further aspect/embodiment, such methods further includes separating the cavernous nerves from the prostate in the absence of heat or electrical energy.

Other aspects, embodiments, and advantages of the present invention will become readily apparent to those skilled in the art and are discussed below. As will be realized, the present invention is capable of other and different embodiments without departing from the present invention. Thus the following description as well as any drawings appended hereto shall be regarded as being illustrative in nature and not restrictive.

### BRIEF DESCRIPTION OF THE DRAWINGS

These and other features and advantages of the invention will be appreciated as the same becomes better understood by reference to the following detailed description when considered in connection with the accompanying drawings wherein:

FIG. 1A shows one embodiment of a laparoscopic instrument in accordance with the present invention having a movable operable end in connection with finder and thumb rings.

FIG. 1B shows another embodiment of a laparoscopic instrument in accordance with the present invention.

FIG. 1C shows another embodiment of a laparoscopic instrument in accordance with the present invention.

FIG. 2A shows an operable end in the form of a modified right angle clamp in accordance with one embodiment of the present invention.

FIG. 2B shows an operable end in the form of a curved dissector in accordance with another embodiment of the present invention.

FIG. 3 shows an example of the keyhole incisions made in performing laparoscopic radical prostatectomy. Typically, 4 to 5 keyhole incisions (about 1 to 1.5 cm) are made across the mid abdomen.

FIG. 4A a cross section of the urethra just distal to the apex of the prostate, demonstrating the outer striated urethral sphincter and the relationship of the neurovascular bundles.

FIG. 4B shows the anatomical relationship of the prostate to the pelvic fascia, pelvic plexus, and neurovascular bundle.

FIG. 5 illustrates dividing the puboprostatic ligaments.

FIG. 6 shows a visual of the field.

FIG. 7 illustrates isolating the dorsal vein complex.

FIG. 8 illustrates dividing the dorsal vein complex

FIG. 9 illustrates oversewing the dorsal vein/urethral sphincter complex.

FIG. 10 illustrates transecting the urethra.

FIG. 11 illustrates the lateral and posterior components of the striated urethral sphincter.

FIG. 12 illustrates dividing the superficial fascia.

FIG. 13 illustrates releasing the lateral pelvic fascia.

FIG. 14 illustrates dividing the lateral pedicies.

FIG. 15 illustrates dividing the bladder neck.

### DETAILED DESCRIPTION

The devices and methods of the present invention are primarily illustrated and described herein by means of devices which have been adapted for use in laparoscopic radical prostatectomy, particularly nerve-sparing radical prostatectomy. One skilled in the art will appreciate that the present invention, although advantageously suited for laparoscopic radical prostatectomy, can be used at any location on or within the body where fine dissection and/or manipulation of delicate tissues is desired. This includes, but is not limited to, urology, general surgery, and gynecology. For example, the present devices and methods will find utility in other laparoscopic procedures such as laparoscopic radical nephrectomy in patients with kidney tumors. Also, one skilled in the art will appreciate that the devices and methods are suitable in laparoscopic surgeries on humans as well as in veterinary procedures. Additionally, one skilled in the art will appreciate that the devices and methods of the present invention also can be suitable adapted so such devices and methods are used in open surgical techniques as well. Thus, the disclosure to follow should be construed as illustrative rather than in a limiting sense.

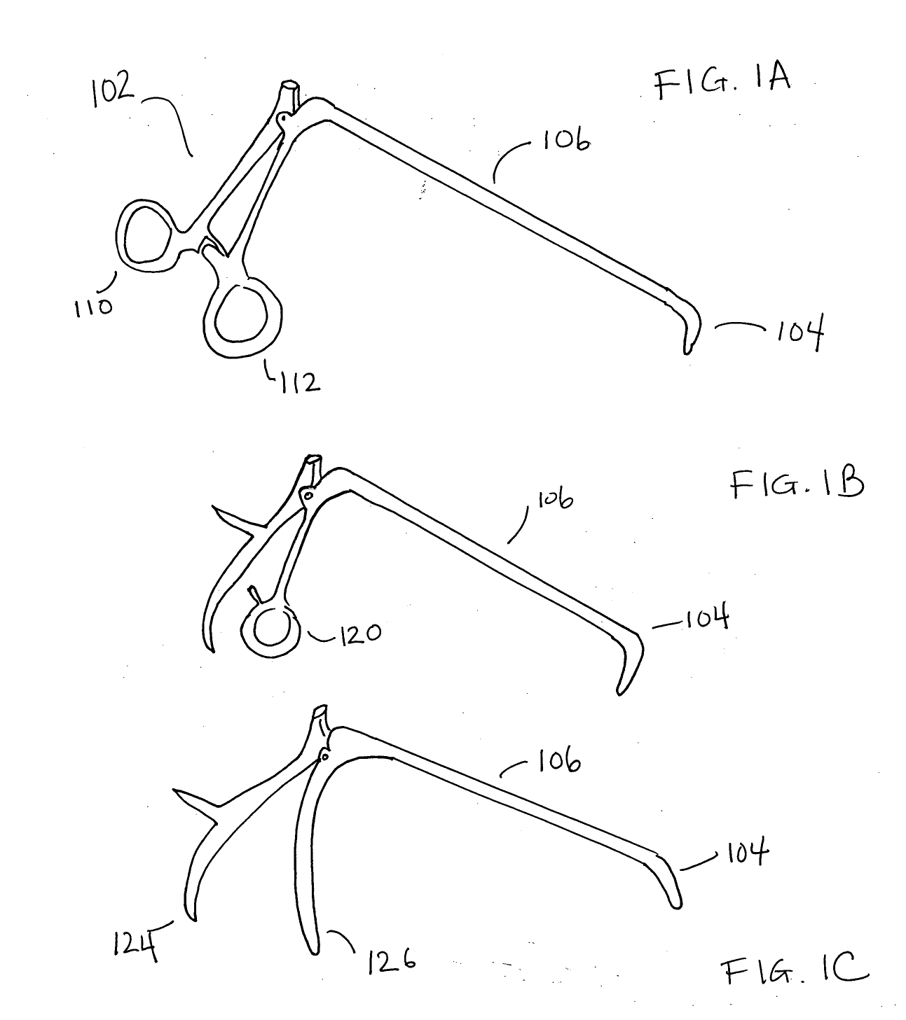

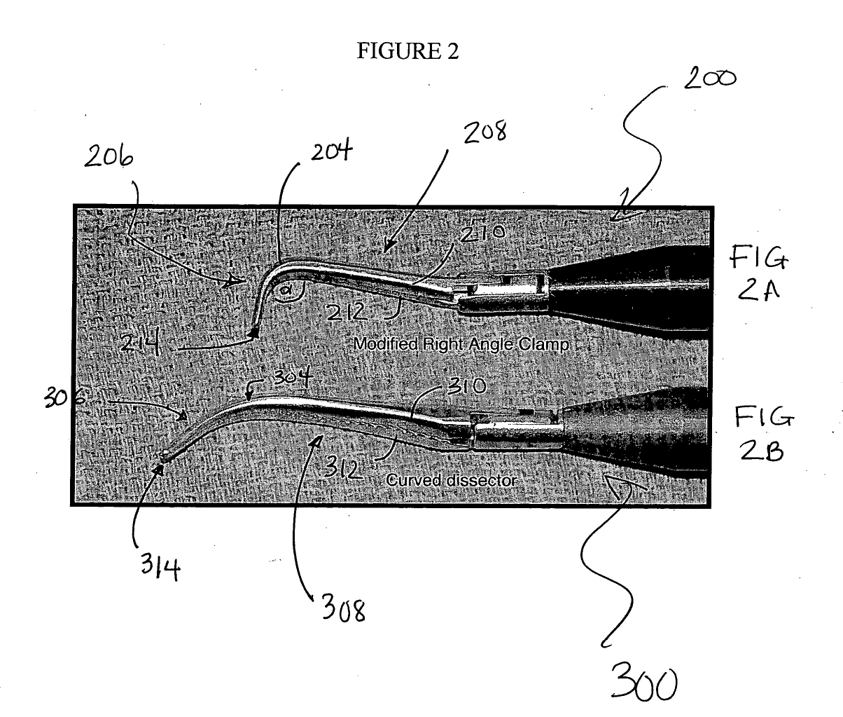

Referring now to the various figures of the drawing wherein like reference characters refer to like parts, there is shown in FIGS. 1A, 1B, 2A and 2B various views of an instrument 100 according to an aspect of the present invention. As shown in FIGS. 1A and 1B, the instrument 100 comprises generally a proximal end 102 defining a handle or hand piece, a distal end 104 defining the operable end of the instrument, and an elongate shaft 106 extending therebetween. (In accordance with conventional practice, “proximal end” designates herein the specified end closest to the medical personnel manipulating the device, and “distal end” designates herein the opposite end placed within a patient.)

The elongate shaft 106 as shown in FIGS. 1A and 1B, has a generally cylindrical shape with a circular cross-section. However, this shall not be construed as limiting the body to such as shape, as it is within the scope of the present invention for other geometric shapes to be used for the shaft 106 such as, for example, an oval, square, hexagon or other cross-sectional shapes. In preferred embodiments, the body includes a smooth outer surface so as to prevent or minimize damage that could result if the shaft contacts tissues or other internal and external structures (particularly in open surgeries) and so as to allow for a smooth manipulation of the shaft 106 within a cannula or sleeve in laparoscopic procedures. Shaft 106 is fabricated from any material as is known to those skilled in the art for use in fabricating such surgical instruments and is preferably lightweight and strong. Such materials are generally bio-compatable materials, which include, but are not limited to, surgical grade stainless steel, anodized aluminum, and polymeric materials and composites. The dimensions of the shaft can vary depending on the type of procedure performed and can be readily determined by one of skill in the art. For example, for laparoscopic applications, suitable diameters of elongate body member are those that will properly fit through and allow for manipulation within the laparoscopic sheath (e.g., from about 5 mm to about 10 mm).

The proximal end 102 is arranged so as to include a handle or hand piece that the surgeon may use to grasp and manipulate the device. When the operable end or the distal end 104 includes arms, jaws or similar movable or grasping mechanisms, the handle suitably is in the form of an actuating handle that is configured and arranged to be manipulated and so such manipulation moves the arms, jaws or movable mechanisms at the operable end. Such actuating handles are well known and thus may embody any of a number of conventional actuating handles as is known to those skilled in the art.

For example, in one preferred embodiment, the actuating handle is adapted to be held in a surgeon's single hand and includes finger and thumb rings 110 and 112 as depicted in FIG. 1A. Finger and thumb rings 110 and 112 can move alone or in combination with respect to each other to enable, for example, grasping of a targeted object. For example, the finger and thumb rings and the arms, jaws or movable mechanisms can move in opposed directions from a center pivot point to enable grasping of a target object. In some embodiments, a single arm or jaw could remain stationary while the other arm or jaw moves toward and away from the stationary arm or jaw. In further embodiments, the arms, jaws or other movable mechanism preferably extends or opens to at least 90° with respect to each other.

In some embodiments, the actuating handle further includes various locking and positioning mechanisms including those known in the art, that allow a user, for example, to move and/or firmly hold the grasping arms at the operable end in a given position. Other conventional arrangements other than finger and thumb rings 110 and 112 could also be used. In one embodiment, a single ring 120 is used in combination with a movable or stationary handle as depicted in FIG. 1B. In another embodiment, the actuating handle includes two handles 124, 126 that can both be movable or individually movable as shown in FIG. 1C.

In other embodiments, wherein the operable end of the device does not include movable parts, for example, where the operable end is a cutting tool or a suction device; the handle or hand piece is a simple handle or hand piece.

In preferred embodiments, the handle or hand piece, for all embodiments shown in FIGS. 1A-1C, further includes a rubber coating, grooves or similar finger grip configuration (e.g., surface preparations or artifacts) to assist or facilitate the surgeon in securely gripping the instrument.

In preferred embodiments, wherein the operable end of the device includes arms, jaws or similar movable or grasping mechanisms that are controlled by an actuating handle, the shaft 106 may suitably comprise a hollow tube that houses push/pull rods in connection with a cam to open and close arms, jaws or similar movable or grasping mechanisms. It is within the scope of the present invention for other conventional mechanisms for activating the arms, jaws or similar movable or grasping mechanisms to be employed in the surgical device of the present invention. In embodiments wherein the operable end of the device includes a suction and/or infusion mechanism, the shaft 105 comprises a hollow tube or includes one or more lumen through which materials can pass between the distal end 104 and proximal end 102 of the device.

The distal end 104 defines the operable end of the instrument and is generally arranged so as to provide any of a number surgical instruments known in the art such as, for example, graspers, retractors, clamps, dissectors, scissors, forceps, biopsy punches, biopsy spoons, hooks and the like.

In one preferred embodiment, the distal end 104 is in the form of a clamp, for example, as shown in FIG. 2A. Clamps are well-known, and the general components of the clamp can be in accordance with conventional clamps. In general, clamps are instruments that are useful during surgical procedures where there is a need to occlude a tubular conduit, such as an artery, vein, or intestine, with little or no damage to the tissues or to grasp such tissue. For example, in a surgical procedure on blood vessels, it often becomes necessary to temporarily stop the flow of blood through the blood vessel.

Clamp comprises generally of a pair of jaws or arms 210, 212 having inner surfaces that are disposed opposite each other and which are in a longitudinally engageable relationship. The jaws or arms 210, 212 are movable such that the inner surfaces may be separated and engaged and held at varying distances from each other with tissue, a vessel or the like held therebetween. In a preferred embodiment, the jaws or arms are moveable such that the distal end 104 of the clamp are placed under or between tissues, nerves and other structures, with the arms or jaws 210 and 212 in a closed position. The arms or jaws 210 and 212 are then separable at varying distances. The separation of the arms or jaws 210 and 212 can be used to cause movement of the tissues, nerves and other structures. In particular, in one embodiment, the distal end 104 is adapted such that it can be inserted between the prostate and the cavernous nerves without damaging the nerves or causing trauma. Subsequently, the arms or jaws 210 and 212 are manipulated to an open position, which causes separation of the cavernous nerves from the prostate. Both the curved and right angled surgical device of the present invention are suitably used for fine blunt dissection of the cavernous nerves off of the prostate surface. As these nerves lie within millimeters of the prostate surface, these fine (e.g., 0.8 mm) tipped dissectors are useful to develop a plane between the nerves and prostate surface to release and preserve them while removing the cancerous prostate gland.

The clamp in accordance with the invention is designed so as to access and handle delicate tissues and nerves, particularly those involved in performing a nerve-sparing laparoscopic radical prostatectomy and, as such, is sized and shaped to facilitate a surgeon's ability to meticulously handle such tissues and nerves. In a preferred embodiment, the clamp includes a proximal portion 208, a bend 204, and a distal portion 206. The proximal and distal portions 208, 206 are preferably generally linear in shape and are at an angle (α) to each other via the bend 204. In a preferred embodiment, wherein the device is used in performing a nerve-sparing laparoscopic radical prostatectomy, in order to access the surgical site, including the neurovascular bundle, angle α ranges from about 60° to about 90°. In a particularly preferred embodiment, angle α is approximately 90°. Unlike conventional clamps, the profile of the clamp is further sized smaller than conventional clamps and, preferably, tapers from proximal end 208 to distal end 206.

In an exemplary embodiment, the clamp has a cross-sectional diameter (or largest cross-sectional dimension for non-circular cross sections) at its smallest location(s) that is no greater than about 2 mm, preferably no greater than about 1.5 mm, preferably no greater than about 1.4 mm, preferably no greater than about 1.3 mm, preferably no greater than about 1.2 mm, preferably no greater than about 1.1 mm, preferably no greater than about 1 mm, preferably no greater than about 0.9 mm, preferably no greater than about 0.8 mm. In a preferred embodiment, wherein the clamp is designed for use in performing a nerve-sparing laparoscopic radical prostatectomy, the clamp has a cross-sectional diameter at its smallest location that is between about 0.5 mm and 1 mm, more preferably between about 0.6 mm and about 0.9 mm, and more preferably between about 0.7 and 0.8 mm. In one embodiment, the clamp has a constant diameter extending from its proximal portion 208, through the bend 204, and to the tip 214 at distal portion 206. In a preferred embodiment, the diameter at the distal portion 208 is larger than that at the tip 214 and tapers gradually from the distal portion 208 to tip 214 as shown in FIG. 2A.

Without being bound by theory, it is believed that such a tapered profile provides added strength at the base of the clamp while providing the ability to provide precise and delicate manipulation and dissection at the distal portion 206 and tip 214 of the device. For example, in one preferred embodiment, the distal portion 208 is positioned under or between tissues and/or nerves (e.g. between the prostate and the cavernous nerves) in a closed position and thereafter, the jaws 210, 212 are opened by manipulating handle so as to separate the tissues and/or nerves (e.g., so as to hold the cavernous nerves away from the prostate so as to allow for fine dissection between the neurovascular bundles and the prostate).

The length of the clamp, which includes the proximal portion 208, bend 204, and distal portion 206 can vary depending on the specific use and surgical procedure and in a particular illustrative embodiment generally ranges between about 10-12 inches and in illustrative embodiments is about 12 inches. The width of the jaws 210, 212 also can vary and in a particular illustrative embodiment is about 3 cm. In a preferred embodiment, the width of the jaws at the distal portion 206 tapers to a range of about 0.6 mm and about 0.9 mm. Such a tapered configuration could be provided by smoothly filing down the tip of a conventional clamp to the desired size.

In an exemplary embodiment, wherein the clamp is designed for use in performing a nerve-sparing laparoscopic radical prostatectomy, the cross-sectional size of the clamp tapers from the proximal end 208 towards tip, the bend 204 provides an angle α of approximately 90°, and the distal portion 206 has a cross-sectional diameter that ranges from about 0.6 mm to about 0.9 mm, preferably about 0.8 mm at the tip 214.

In another preferred embodiment, the distal end 104 defines that of a dissector, particularly a curved dissector generally depicted as 300 in FIG. 2B. Dissectors are well-known, and the general components of the dissector can be in accordance with conventional dissectors. Dissectors comprise generally of a pair of jaws 310, 312 having inner surfaces that are disposed opposite each other and which are in a longitudinally engageable relationship. The jaws 310, 312 are movable such that the inner surfaces may be separated and engaged and held at varying distances from each other. Jaws 310, 312 typically have sharp inner teeth located along their inner surfaces for dissection of tissues.

The dissector in accordance with the invention is designed so as to access, dissect, and handle delicate tissues and nerves, particularly those involved in performing a nerve-sparing laparoscopic radical prostatectomy and, as such, is sized and shaped to facilitate a surgeon's ability to meticulously handle and dissect such tissues and nerves. In a preferred embodiment, the dissector includes a proximal portion 308, a bend 304, and a distal portion 306. The proximal and distal portions 308, 306 are preferably generally linear in shape and are at an angle (α) to each other via bend 304. In a preferred embodiment, wherein the device is used in performing a nerve-sparing laparoscopic radical prostatectomy, in order to access the surgical site, including the neurovascular bundle, angle α ranges from about 60° to about 135°. In a particularly preferred embodiment, angle α is approximately 90° and in another preferred embodiment, angle α is approximately 135°. Unlike conventional dissectors, the profile of the present dissector is further sized smaller than conventional dissectors and, preferably, tapers from proximal end 308 to distal end 306.

In an exemplary embodiment, the dissector has a cross-sectional diameter (or largest cross-sectional dimension for non-circular cross sections) at its smallest location(s) that is no greater than about 2 mm, preferably no greater than about 1.5 mm, preferably no greater than about 1.4 mm, preferably no greater than about 1.3 mm, preferably no greater than about 1.2 mm, preferably no greater than about 1.1 mm, preferably no greater than about 1 mm, preferably no greater than about 0.9 mm, preferably no greater than about 0.8 mm. In a preferred embodiment, wherein the dissector is designed for use in performing a nerve-sparing laparoscopic radical prostatectomy, the dissector has a cross-sectional diameter at its smallest location that is between about 0.5 mm and 1 mm, more preferably between about 0.6 mm and about 0.9 mm, and more preferably between about 0.7 and 0.8 mm. In one embodiment, the clamp has a constant diameter extending from its proximal portion 308, through the bend 304, and to the tip 314 at distal portion 306. In a preferred embodiment, the diameter at the distal portion 308 is larger than that at the tip 314 and tapers gradually from the distal portion 308 to tip 314 as shown in FIG. 2B.

Without being bound by theory, it is believed that such a tapered profile provides added strength at the base of the dissector while providing the ability to provide precise and delicate manipulation and dissection at the distal portion 306 and tip 314 of the device. The length of the clamp, which includes the proximal portion 308, bend 304, and distal portion 306 can vary depending on the specific use and surgical procedure, but generally ranges from about 10 to 12 inches and in illustrative embodiments is about 12 inches. The width of the jaws 310, 312 can also vary and in a particular illustrative embodiment is about 3 cm. In a preferred embodiment, the width of the jaws at the distal portion 306 tapers to a range of about 0.6 mm and about 0.9 mm. Such a tapered configuration could be provided by smoothly filing down the tip of a conventional dissector to the desired size.

In exemplary embodiments, wherein the dissector is designed for use in performing a nerve-sparing laparoscopic radical prostatectomy, the cross-sectional size of the dissector tapers from the proximal end 308 towards tip, the bend 304 provides an angle α of approximately 90° in one embodiment and approximately 135° in another embodiment, and the distal portion 306 has a cross-sectional diameter that ranges from about 0.6 mm to about 0.9 mm, preferably about 0.8 mm at the tip 314.

The instruments may be fabricated of conventional materials used in forming surgical instruments, for example, surgical grade stainless steel, anodized aluminum, and polymeric materials and composites. Preferably, the instruments are reusable and easily cleaned and sterilized. In some embodiments, the components can be at least partially disassembled for ease in cleaning and sterilizing. In other embodiments, all or portions of the instruments are disposable.

The dimensions of the device can vary and depend on factors such as the type of procedure, the age and size of the subject. For laparoscopic procedures, the length of the instruments are generally greater than for open surgeries. For example, for laparoscopic radical prostatectomies, the length of the instrument can range from about 10-12 inches whereas for open radical prostatectomies, the length of the instrument can range from about 6-10 inches. These dimensions are given for illustrative purposes only and are not meant to be limiting.

The present invention also includes kits that comprise one or more surgical devices of the present invention, preferably packaged in sterile condition. Such kits may include written instructions for use of the device and other components of the kit.

Methods of the present invention comprise performing a nerve-sparing radical prostatectomy by using the surgical instruments/devices of the present invention to gently tease away the cavernous nerve fibers from the prostate surface. Meticulous dissection with such surgical present instruments/devices allows for development of the tissue plane that exists between the nerves and prostate using blunt dissection.

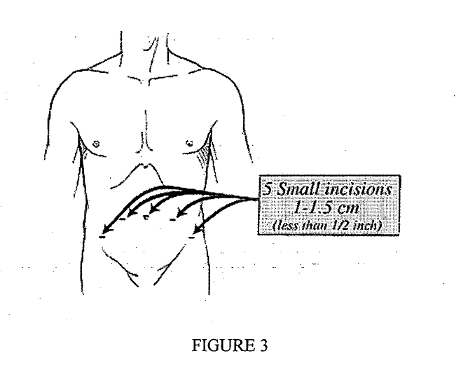

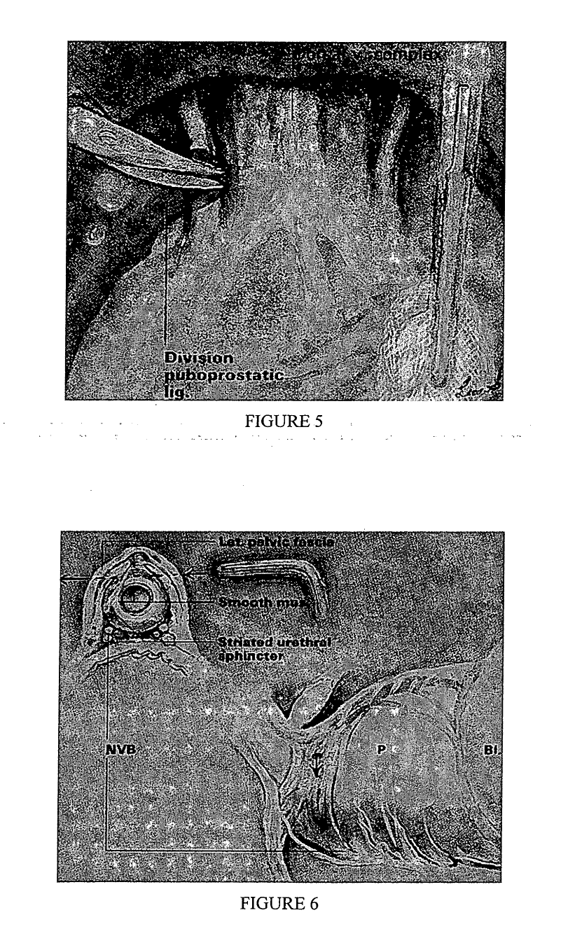

There is shown illustratively in FIGS. 3-15, the general steps for performing a nerve-sparing radical prostatectomy, however, the methods of the present invention shall not be construed as being particularly limited to that being shown or illustrated. Further such methods of the present invention also shall embody or include the steps described below in the illustrative examples not otherwise described below in connection with FIG. 13-15. In particular, such methods also shall include performing the actions described hereinafter without using thermal or electrical energy to achieve hemostasis (e.g., without using electrocatery).

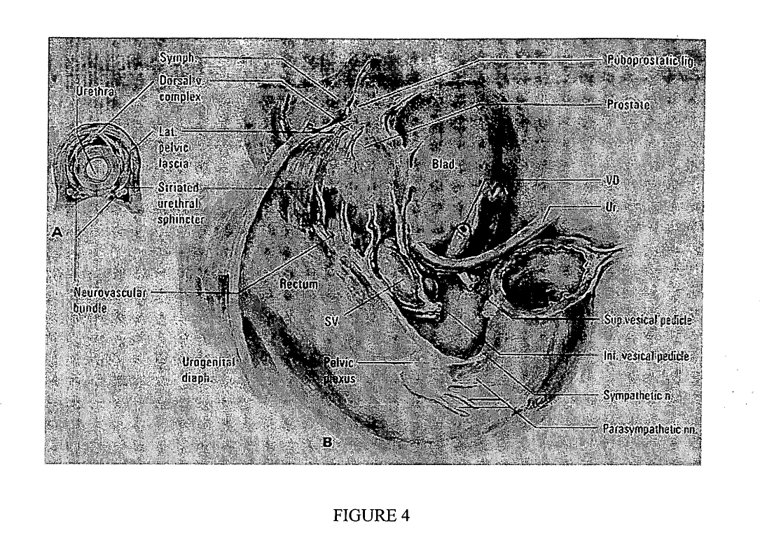

As shown in FIG. 3, the surgical site would be accessed through 4 or 5 small incisions across the mid-abdomen. FIG. 4A shows a cross section of the urethra distal to the apex of the prostate, demonstrating the outer striated urethral sphincter and the relationship of the neurovascular bundles. FIG. 4B shows the anatomical relationship of the prostate to the pelvic fascia, pelvic plexus, and neurovascular bundle. Once the surgical site is accessed, tension is placed on the puboprostatic ligaments and the ligaments incised laterally. The superficial branch of the dorsal vein is divided.

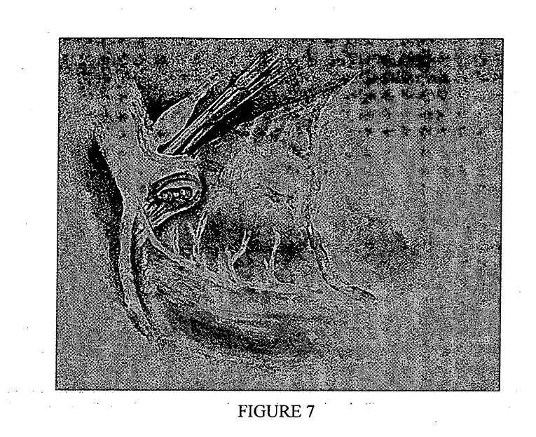

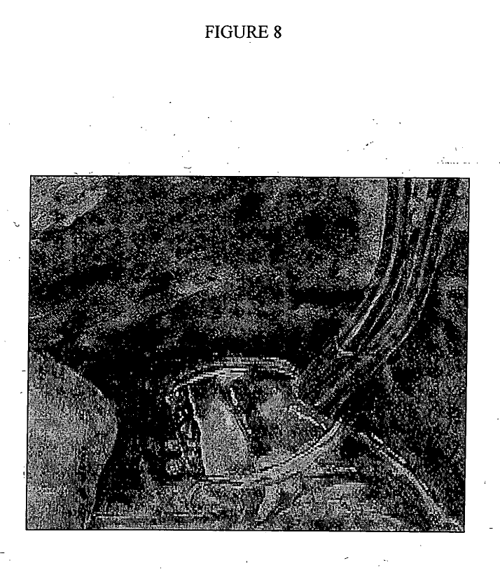

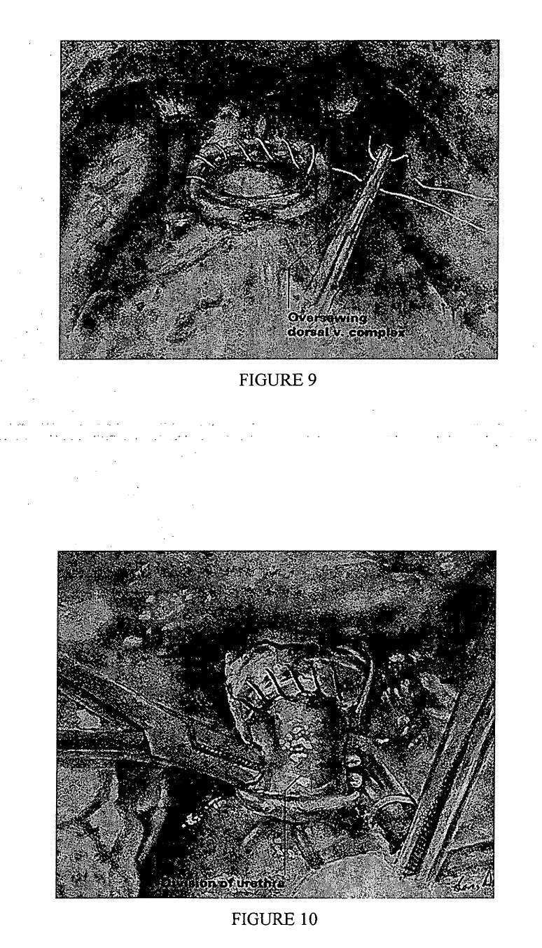

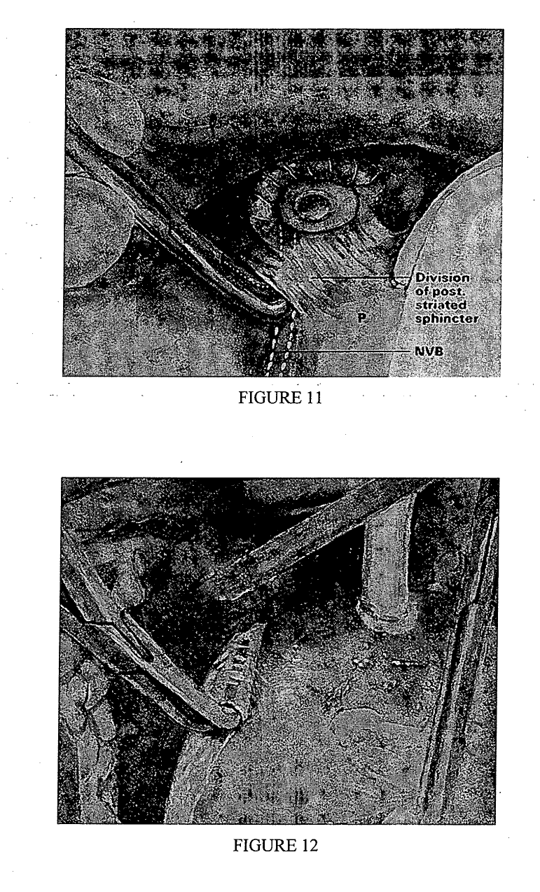

Next, as shown in FIG. 7, the lateral wall of the urethra is identified and the lateral pelvic fascia is gently perforated by a clamp. The clamp is then positioned beneath the dorsal vein complex and surrounding striated urethral musculature as shown in FIG. 8. The dorsal vein is ligated and divided or divided without ligation. As shown in FIG. 9, the dorsal vein/striated urethral sphincter complex is oversewn horizontally and any large venous channels at the posterolateral edges may be oversewn. As shown in FIG. 10, the urethra is gently separated from the lateral and posterior portions of the striated sphincter and the anterior surface of the urethra is divided. As shown in FIG. 12, the superficial lateral pelvic fascia on the lateral surface of the prostate is released, thereby releasing the prostate, making it more mobile, and exposing the location of the neurovascular bundle. In accordance with the present methods, the present instruments, particularly the fine tips of the instruments, are sized and shaped such that they can be inserted under the lateral pelvic fascia to begin to develop a plane between the nerve bundles and prostate gland. In FIG. 13, beginning at the apex of the prostate, the lateral pelvic fascia is gently released posteriorly from the edge of the prostate, using a right angle clamp. A small arterial branch is clipped and divided. Having released the neurovascular bundle at the apex, the dissection continues to the midprostate.

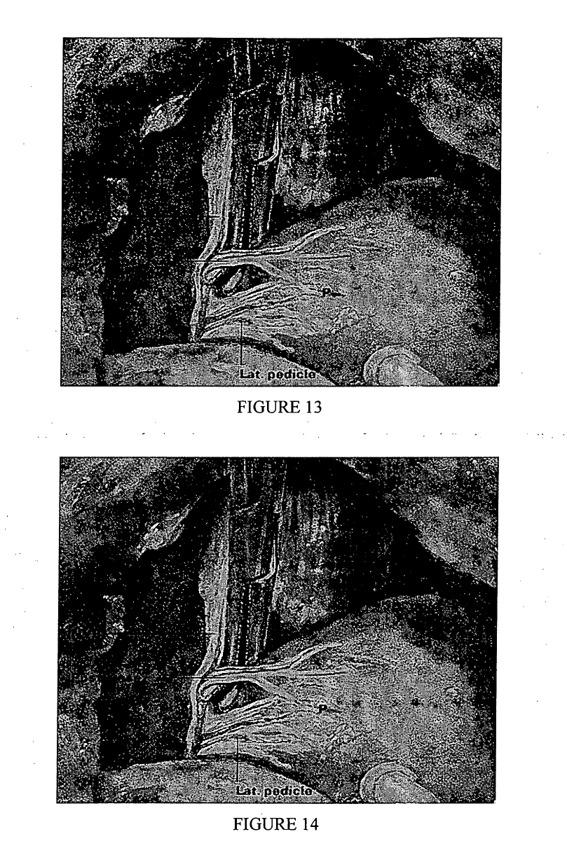

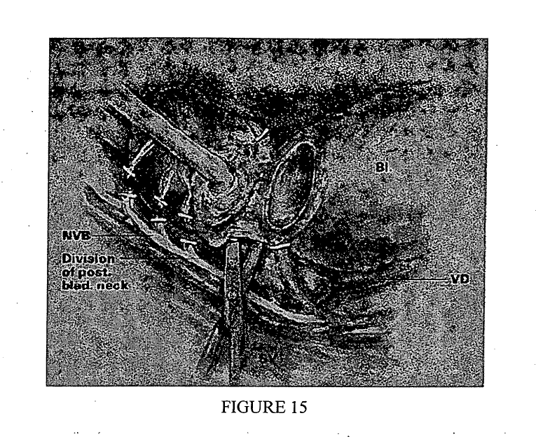

As shown in FIG. 14, once the neurovascular bundles have been released on both sides, the attachment of Denonvilliers' fascia to the rectum is divided in the midline, maintaining all layers of Denonvilliers' fascia on the prostate. At this point, a prominent arterial branch running from the neurovascular bundle to the posterior surface of the prostate is identified. By dividing this posterior branch, the neurovascular bundle falls posteriorly, reducing the chance for injury during division of the lateral pedicies. The lateral pedicies are next divided on the lateral surface of the seminal vesicles without ligation. The specimen is then removed as shown in FIG. 15.

All documents mentioned herein are incorporated by reference herein in their entirety. The following non-limiting example is illustrative of the invention.

### EXAMPLE 1

177 patients underwent LRP performed by one of two surgeons. The preoperative potency status was determined using an abridged version of the Inter-national Index of Erectile Function and the Expanded Prostate Cancer Index Composite. Postoperative potency was assessed at 3, 6, and 12 months after surgery and was defined as the patient-reported ability to achieve an erection sufficient for penetration and intercourse with or without sildenafil citrate. This information was obtained from written answers to item number 63 of the Expanded Prostate Cancer Index Composite (“during the last 4 weeks, how often did you have sexual intercourse?”). Any answer other than “not at all” was coded as an affirmative.

To facilitate meticulous neurovascular bundle dissection and simulate the open surgical techniques used at our institution, two laparoscopic instruments were specially designed. These included a 10-mm right-angle dissector and a 10-mm curved tonsil dissector (Aesculap, Center Valley, Pa.). The tips of these two instruments were modified to 0.8 mm in width to allow for fine dissection between the neurovascular bundles and the prostate (FIGS. 2A and 2B).

Transperitoneal LRP was performed according to the Montsouris technique. During dissection of the lateral aspect of the seminal vesicles, Hemolock clips (Week Closure Systems, Re-search Triangle Park, N.C.) were used to secure the blood vessels in lieu of electrocautery or use of ultrasonic shears, avoiding the use of energy sources around the nearby neurovascular bundles. After dissection of the seminal vesicles and vasa deferentia and the incision of Denonvilliers' fascia, the space of Retzius was entered and the anterior aspect of the prostate exposed. The endopelvic fascia was opened sharply, and the puboprostatic ligaments were divided. The dorsal venous complex was ligated using 2-0 polyglactin suture loaded on a GS-21 needle. A traction suture was placed along the anterior mid-portion of the prostate, allowing the assistant to reflect the prostate laterally and expose the contralateral lateral prostatie fascia. As in open RRP, the lateral prostatic fascia was incised with scissors, and the plane between the fascia and prostatic capsule was developed using the modified fine right-angle dissector. This plane of dissection was carried distally and proximally along the lateral aspect of the prostate using mainly blunt dissection with the right-angle dissector and “cold” incision with laparoscopic scissors. A neurovascular bundle “groove” was developed by maintaining close dissection along the prostatic capsule and continuing the plane in a posterolateral direction. Near the apex, the modified curved dissector was useful in separating the neurovascular bundle from the posterolateral aspect of the prostate gland. Similar nerve dissection was then performed on the contralateral side.

After completion of the lateral dissection of both neurovascular bundles, the bladder neck was divided, and the seminal vesicles and vasa deferentia were brought through the opening of the posterior bladder neck and placed on gentle anterior traction. Large (10-mm) Hemolock clips were used to secure the prostatic pedicles. Using the previously dissected neurovascular bundle groove as a guide, antegrade dissection of the neurovascular bundle was performed. A combination of sharp and blunt dissection gently teases the nerve bundle off of the posterolateral surface of the prostate. Small 5-mm Hemolock clips were used, as needed, to secure the vessels traversing between the neurovascular bundle and the prostate, and energy sources were avoided. After bilateral antegrade dissection of the neurovascular bundles was carried out as far to the apex as possible, the urethra was divided sharply, and the distal aspect of the neurovascular bundle was spared using retrograde dissection with the modified right-angled clamp. Preserved bilateral neurovascular bundles were easily seen after complete removal of the prostate gland.

The specimen was placed in an endoscopic bag and extracted through an extension of the umbilical trocar site at the end of the procedure. The vesicourethral anastomosis was performed using interrupted 2-0 polyglactin sutures on a GU-46 needle. Great care was taken to avoid incorporating the neurovascular bundle into the anastomotic sutures, especially at the 5-o'clock and 7-o'clock positions along the urethra. Both an 18F silicone urethral catheter and a closed-suction pelvic drain were placed. After removal of the prostate specimen, inspection of the posterolateral surface revealed minimal tissue, indicating excellent preservation of the neurovascular bundles.

Of the 177 LRPs, 173 (97.7%) were completed laparoscopically. Blood loss was minimal (less than 300 mL). Bilateral nerve preservation was performed in 89 patients (51.4%), unilateral preservation in 57 (32.9%), and non-nerve-sparing LRP in 27 (15.6%). The positive margin rate using this technique was 11.3% overall (20 of 177) and was 4.7% for patients with pathologic Stage T2 disease (7 of 148). With a mean follow-up of 13.2 months (range 3.4 to 33.3), 40% of patients who underwent LRP reported a return of erections sufficient for sexual intercourse at 6 months and 48% at 1 year. Stratifying the results by those who re-ported normal preoperative erections (International Index of Erectile Function score of more-than 21) and who had bilateral nerve preservation, the patient-reported potency was greater than in unselected patients (Table 1). Finally, for all potent men who reported engaging in sexual intercourse to reduce peritoneal tears and vascular injuries and reproduce more precisely the open procedure while keeping the same landmarks, which facilitates possible conversion, especially during the learning phase.

It was demonstrated that the nerve-sparing LRP technique and instrumentation was able to replicate established open surgical principles of anatomic nerve-sparing RRP. The techniques minimize the potential for cavernous nerve damage from electrical energy or heat, and early functional outcomes are comparable to the results obtained with open RRP.

TABLE 1
Potency outcomes after LRP. Percentage of patients engaging in sexual
intercourse 3, 6, and 12 months after LRP shown. Data derived from item
number 63 of Expanded Prostate Cancer Index Composite questionnaire.
Results stratified by preoperative potency status as follows: white bars
Indicate all patients; gray bars indicate patients with preoperative
International Index of Erectile Function score of more than 21 who
underwent bilateral nerve sparing; and black bars indicate patients
engaging in intercourse preoperatively who underwent bilateral
nerve sparing.

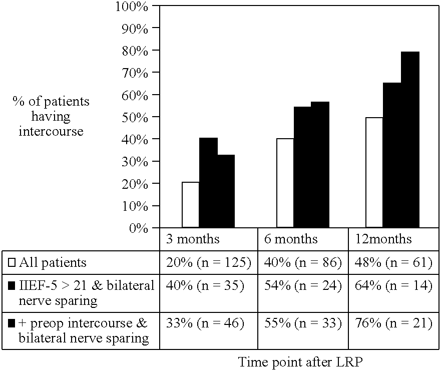

Although the instruments and methods of the present invention are primarily illustrated and described herein by means of instruments which have been adapted for performing laparoscopic radical prostatectomy on humans, it will be appreciated by those skilled in the art that such instruments and methods also are adaptable for use in other particularly delicate surgical procedures (both open and laparoscopic) as well as in performing various veterinary surgeries. Further, while the instruments and methods are primarily illustrates and described in connection with clamps and dissectors, other instruments (e.g. various laparoscopic and open surgery instruments such as graspers, scissors, forceps, biopsy punch, biopsy spoon, and hooks) could likewise be provided as described herein. Further, while a preferred embodiment of the invention has been described using specific terms, such description is for illustrative purposes only, and it is to be understood that changes and variations may be made without departing from the spirit or scope of the following claims.

What is claimed is:

1. A surgical instrument suitable for use in performing nerve-sparing radical prostatectomy comprising:
an elongate shaft having a proximal end and a distal end;
a handle at the proximal end of the shaft;
an operable end at the distal end of the shaft comprising at least two arms that are disposed opposite each other, the two arms having inner surfaces in longitudinal engagement with each other;
the operable end having a proximal portion, a distal portion, and a curved portion between the proximal portion and distal portion
wherein the distal portion tapers to a diameter of less than about 2 mm.

2. The instrument of claim 1, wherein the distal portion tapers to a diameter of less than about 1.5 mm.

3. The instrument of claim 1, wherein the distal portion tapers to a diameter of less than about 1.4 mm.

4. The instrument of claim 1, wherein the distal portion tapers to a diameter of less than about 1.3 mm.

5. The instrument of claim 1, wherein the distal portion tapers to a diameter of less than about 1.2 mm.

6. The instrument of claim 1, wherein the distal portion tapers to a diameter of less than about 1.1 mm.

7. The instrument of claim 1, wherein the distal portion tapers to a diameter of less than about 1 mm.

8. The instrument of claim 1, wherein the distal portion tapers to a diameter of from about 0.5 mm to about 0.9 mm.

9. The instrument of claim 1, wherein the distal portion tapers to a diameter of about 0.8 mm.

10. The instrument of claim 1, wherein the curved portion forms an angle α between the proximal portion and distal portion ranging from about 160° to about 90°.

11. The instrument of claim 10, wherein the operable end comprises a clamp and angle α is approximately 90°.

12. The instrument of claim 10, wherein the operable end comprises a dissector and angle α is approximately 135°.

13. The instrument of claim 11 or 12, wherein the distal portion tapers to a diameter of from about 0.5 mm to about 0.9 mm.

14. The instrument of claim 11 or 12, wherein the distal portion tapers to a diameter of about 0.8 mm.

15. The instrument of claim 1, wherein the handle includes one or more movable finger and thumb rings in connection with the arms or jaws, whereby manipulation of one or more finger and thumb rings changes the distance between inner surfaces of the arms or jaws.

16. The instrument of claim 1, wherein the instrument is a laparoscopic instrument.

17. A method for performing nerve-sparing radical prostatectomy comprising:
providing a surgical instrument comprising: an elongate shaft having a proximal end and a distal end, an operable end at the distal end of the shaft comprising at least two arms that are disposed opposite each other, the two arms having inner surfaces in longitudinal engagement with each other, the operable end having a proximal portion, a distal portion, and a curved portion between the proximal portion and distal portion, wherein the distal portion tapers to a diameter of less than about 2 mm;
providing the two arms with their inner surfaces in longitudinal engagement;
inserting the distal portion of the operable end between the prostate and the cavernous nerves;
manipulating the arms so as to provide a distance between their inner surfaces, thereby separating the prostate from the cavernous nerves; and
dissecting the cavernous nerves from the prostate.

18. The method of claim 17 wherein the surgical instrument is in the form of a laparoscopic instrument and the method further comprises:
forming a plurality of incisions in the mid abdomen;
inserting a plurality of laparoscopic sleeves through the incisions; and
inserting the surgical instrument into the surgical site via a laparoscopic sleeve.

19. The method of claim 17 wherein the cavernous nerves are separated from the prostate in the absence of heat or electrical energy.
